(19) United States
(12) Patent Application Publication
(10) Pub. No.: US 2022/0072165 A1
(43) Pub. Date: Mar. 10, 2022

(54) RADIODRUG FOR DIAGNOSTIC/THERAPEUTIC USE IN NUCLEAR MEDICINE AND RADIO-GUIDED MEDICINE

(71) Applicants: UNIVERSITÀ DEGLI STUDI DI ROMA "LA SAPIENZA", Roma (IT); FONDAZIONE POLICLINICO UNIVERSIT ARIO AGOSTING GEMELLI IRCCS, Roma (IT); UNIVERSITÀ CATTOLICA DEL SACRO CUORE, Milano (IT)

(72) Inventors: Riccardo FACCINI, Roma (IT); Elena SOLFAROLI CAMILLOCCI, Roma (IT); Dante ROTILI, Roma (IT); Alessia CIOGLI, Roma (IT); Antonella CARTONI, Roma (IT); Ilaria FRATODDI, Roma (IT); Iole VENDITTI, Roma (IT); Alessandro GIORDANO, Roma (IT); Daria MACCORA, Roma (IT); Germano PEROTTI, Roma (IT); Teresa SCOTOGNELLA, Roma (IT)

(21) Appl. No.: 17/417,989

(22) PCT Filed: Jan. 8, 2020
(86) PCT No.: PCT/IB2020/050105

§ 371 Date: Jun. 24, 2021

(30) Foreign Application Priority Data
Jan. 8, 2019 (IT) 102019000000202

(51) Int. Cl. A61K 51/04; A61P 35/00

(52) U.S. Cl. CPC A61K 51/0497; A61K 51/048; A61P 35/00; A61K 51/0478; A61K 51/0482

(57) ABSTRACT

Radio-drug suitable for performing radio-guided surgery, imaging diagnostics, radio-metabolic therapy.

### STATE OF THE ART

The radio-drugs (also called radioactive tracers or radio-tracers) are medicines including one or more radioisotopes capable of emitting radiation.

The radio-tracers can be used in the radio-guided surgery (RGS) of tumors, i.e. a surgical technique which makes use of radiation emitted by radioactive tracers to discriminate the cancerous tissue from surrounding healthy organs, thus improving the tumor removal process. The radio-tracer is preferentially captured by the neoplastic cells, and such neoplastic cells are identified by a device, called probe, capable of detecting the radiation emitted by the radio-tracer. RGS allows the surgeon to evaluate in real time the completeness of tumor resection, reducing to the minimum the amount of healthy tissue removed.

RGS may be crucial for the survival of oncological patients for whom removing the tumor mass is the only therapeutic option.

RGS is conventionally based on a combination of radio-drugs emitting γ radiation and specific probes sensitive to such radiation. Although RGS with γ radiation is widely validated, current clinical applications of this technique are limited to colon cancer (radio-immune-guided surgery), sentinel lymph node mapping in malignant melanoma and breast carcinoma, identification of parathyroid adenoma and some bone tumors. The major limitation of RGS with γ radio-emitters is in fact the high penetration power of the γ rays. Since these can go through large quantities of tissue, each possible uptake of radio-tracer by the healthy tissues surrounding the tumor can result in not-negligible background radiation (comparable to or higher than the signal coming from the tumor lesions) which may preclude the application of the technique. For the same reason, the medical staff can be exposed to a substantial dose of radiation, unless a very low dose of radio-tracer is administered. Therefore, RGS with γ radiation cannot be applied to many tumors, such as for example brain tumors (given the high uptake of the healthy brain), abdominal tumors (especially if close to kidney, bladder and liver, because of the big tracer uptake by such organs) and pediatric tumors (where, due to the small size, the distances between the organs are very short).

RGS has been applied also in combination with β⁺ radiation emitters. However, the implementation and use of the probes for detecting the β⁺ radio-drugs emitters are complicated, because such probes must shield the γ rays which however are produced when the β⁺ positron emitted is annihilated by interaction with the electrons of body tissues. For this reason, the development of RGS with β⁺ has never passed the pre-clinical phase.

Very recently, in order to overcome the aforementioned limits of RGS with γ or β⁺ radiation, the use in RGS of radio-tracers which are pure electron emitters, i.e. β⁻ particles, has been proposed. The β⁻ radiation penetrates only a few millimeters into the tissues and may be, depending on the radionuclides used, substantially free of γ contamination. Furthermore, the specific β⁻ probes are compact and easy to handle. Additionally, RGS with β⁻ is also effective in administering much lower amount of β⁻ radio-tracer with respect to the amount conventionally administered of γ or β⁺ radio-tracers, thus allowing to operate with a lower background by the healthy tissues surrounding the tumor lesion and providing a clearer delineation of the margins of the same. This allows to extend the field of application of RGS also in cases with a big uptake by the surrounding healthy organs. The lower dose absorbed and the short range of action of the electrons (β⁻ radiation) finally imply almost negligible exposure to radiation by the medical and paramedical staff.

The validation and applicability of the β⁻ tracers radio-guided surgery (β⁻-RGS) strictly depends on the availability of β⁻ emitting radio-drugs, which is currently very limited. Therefore, there is the need to develop radio-drugs capable of emitting β⁻ radiation showing a preferential uptake by tumor tissues/lesions, which must undergo resection by RGS and which show an optimal pharmacokinetic profile for the administration to humans.

### OBJECTS OF THE INVENTION

Object of the present invention is to provide a radio-drug, in particular a β⁻ emitting radio-drug, suitable for performing radio-guided surgery, imaging diagnostics, radio-metabolic therapy, as well as other applications.

Object of the present invention is also to provide a composition comprising said radio-drug, in particular β⁻ emitting radio-drug.

Finally, object of the present invention is to provide uses and applications for said radio-drug, particularly β⁻ emitting radio-drug.

Object of the present invention is also to provide a radio-drug and a composition comprising it, which, by suitably changing the radio-metal, can be used in PET and SPECT imaging diagnostics.

### DESCRIPTION OF THE INVENTION

These and other objects are achieved by the compound of Formula I:

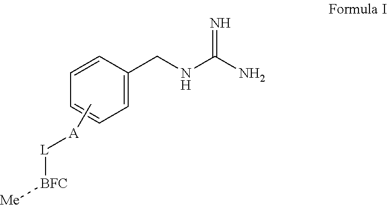

wherein A is an anchoring portion bonded to the meta or para position of benzylguanidine (BG) capable of bonding L, L is a linker portion, BFC is a bifunctional chelator, and Me is a radiation-emitting or non-radiation-emitting metal cation. Particularly preferred is a β⁻ radiation-emitting metal cation.

Alternatively, said metal cation is a γ or β⁺ radiation-emitting metal cation. In particular, with reference to Formula I:

A is an anchoring portion which is chosen from a carboxamide, anilide, ether, amine and sulfonamide function, preferably it is a carboxamide bonded to the meta or para position of BG;

L is a linker portion which is a diaminoalkyl chain or diaminopolyethylene glycol chain, preferably it is an ethylenediamine chain;

BFC is a bifunctional chelator chosen from the group comprising tetraazacyclo DOdecan-Tetra Acetic acid (DOTA), 1,4,7-triazacycloNOnane-N,N′,N″-TriAcetic acid (NOTA), and DiethyleneTriaminoPentAcetic acid (DTPA), preferably it is DOTA;

Me is a radiation-emitting or non-radiation-emitting metal cation. In case the metal cation is a pure β⁻ emitter, preferably it is ⁹⁰Y³⁺. In case the radio metal is a non-pure β⁻ emitter, for example ¹⁷⁷Lu³⁺ is chosen. In case the metal cation is a β⁺ emitter, for example ⁸⁶Y³⁺ and ⁶⁸Ga³⁺ are chosen. In case the metal cation is a γ emitter, for example. ¹¹¹In³⁺ is chosen. In case the metal cation is a non-radiation-emitting cation, preferably ⁸⁹Y³⁺ is chosen.

An object of the invention is a compound having Formula I, wherein A is an anchoring portion chosen from the group consisting of amide, anilide, ether, amine and sulfonamide, bonded to the meta or para position of benzylguanidine (BG); L is a linker portion chosen from the group consisting of diaminoalkyl chains with lengths ranging from 2 to 6 methylene units and diaminopolyethylene glycol chains with lengths ranging from 2 to 6 ethylene glycol units; BFC is a bifunctional chelator chosen from the group consisting of DOTA, NOTA, TETA and DTPA; and Me is a radiation-emitting metal cation chosen from the group consisting of ⁹⁰Y³⁺, ¹⁷⁷Lu³⁺, ⁸⁶Y³⁺, ⁶⁸Ga³⁺ and ¹¹¹In³⁺ or else a non-radiation-emitting metal cation such as ⁸⁹Y³⁺.

As it will be observable in the experimental section, the compound of Formula I, i.e. the compound of the invention, has proven to be surprisingly effective, mostly as pure β⁻ radio-tracer, for the hyper-secretory catecholamine tumors which overexpress the norepinephrine transporter (NET), as well as for other applications (for example, therapeutic applications and, by suitably changing the radio metal, also diagnostic applications).

The compound of the invention consists of five main components (which will be described in detail below): a benzylguanidine portion as selectivity function for some types of tumor cells, a β⁻ radiation-emitting metal cation Me, a bifunctional chelator (BFC), a linker portion L (i.e., a linker) and an anchoring portion A, the latter two useful to bond the benzylguanidine portion to the bifunctional chelator BFC.

The compound of the invention is a pure β⁻ radio-emitter and is the preferential substrate of the axonal carrier of norepinephrine (NE). Therefore, the compound of the invention will be preferentially captured by the tumor cells which overexpress NET (hyper-secreting catecholamines). Among these there are many neuroendocrine tumors, such as pheochromocytomas, paragangliomas, carcinoid tumors and neuroblastomas, which will take significant advantages from the complete resection by β⁻-RGS, as well as monitoring by imaging diagnostics which exploits the positron emission tomography (PET). The preferential uptake by the tumor cells overexpressing NET could be due to the structural analogy of the benzylguanidine contained in the compound of the invention with the neurohormone NE, which is the endogenous substrate of NET.

The compound of the invention, when the metal cation is suitably chosen, is capable of emitting β⁻ radiation as it can have a trivalent radio-metal (Me³⁺) which, by decay, emits β⁻ radiation. Me more preferably is ⁹⁰Y³⁺ for its half-life characteristics (64 hours), energy spectrum (˜2 MeV) and absence of concomitant γ decays (pure emitter) making it perfect for β⁻-RGS. The metal cation ⁹⁰Y³⁺ is conventionally used in nuclear medicine for therapeutic purposes, such as for example in receptor radio-metabolic therapy with marked peptides (PRRT, Peptide Receptor Radionuclide Therapy) which exploits the β⁻ emission. The metal cation ⁹⁰Y³⁺ has never been associated with the use as β⁻ radio-tracer, useful for the localization of tumor lesions during RGS methods of NET-positive tumors. Therefore, the compound of the invention for the first time proves the effectiveness of the metal cation ⁹⁰Y³⁺, included in the compound of Formula I, as β⁻ radio-tracer in the radio-guided surgery (RGS) of the tumors overexpressing NET.

In an alternative embodiment, Me can be a metal cation which doesn't emit radiation, for example ¹¹¹Y³⁺. The compound of the invention comprises such metal and thus is a “cold” compound, i.e. non-marked, and is useful for the characterization of the compound of the invention, as it has chemical-physical and chemical-biological properties, for example the pharmacokinetic and pharmacodynamic properties, coinciding with those of the marked compounds of the invention. The trivalent metal cation Me can alternatively be ¹⁷⁷Lu³⁺ for teragnostic applications (radio-metabolic therapy exploiting the β⁻ emission and contemporary SPECT imaging exploiting the γ emission), ⁸⁶Y³⁺ and ⁶⁸Ga³⁺ for PET imaging applications exploiting the β⁺ emission, ¹¹¹In³⁺, for SPECT imaging applications exploiting the γ emission.

The metal cation Me is bonded to the compound of the invention by coordination bond thanks to the bifunctional binder BFC (“bi-functional chelator”). By BFC is meant a molecule having the dual function of chelating a metal cation and contemporary of advantageously and covalently bonding a functional portion. In the case of the compound of the invention, the functional portion is benzylguanidine; such functional portion is bonded to BFC by an anchoring portion A and a linker portion L. The preferred bifunctional chelator BFC according to the present invention is DOTA (tetraaza-cyclododecane tetra-acetic acid). Other bifunctional chelators BFC advantageous for the trivalent metal cations object of the present invention (⁹⁰Y³⁺, ⁶⁸Ga³⁺, ⁸⁶Y³⁺, ⁹⁸Y³⁺, ¹⁷⁷Lu³⁺ and ¹¹¹In³⁺) as they are better in terms of marking efficiency and overall chemical/metabolic stability of the resulting chelate, are the linear or cyclic analogues of DOTA, as well as 1,4,7-triazacycloNOnane-N,N′,N″-TriAcetic acid (NOTA), TEtraazacyclotetradecane-1,4,8,11-TetraAcetic acid (TETA) and DiethyleneTriammoPentAcetic acid (DTPA).

The linker portion L is any portion avoiding the interference (for example, the steric and/or functional interference) between the metal chelator and benzylguanidine. Preferably, when BFC is DOTA, the linker portion L is an ethylenediamine portion. Other linker portions L can be used according to the compound of the invention, for example diaminoalkyl chains with lengths ranging from 2 to 6 methylene units, diaminopolyethylene glycol chains with lengths ranging from 2 to 6 ethylene glycol units. The linker portion L is preferably covalently bonded both to the bifunctional chelator BFC and anchoring portion A, for example by amide bonds when said linker portion L is an ethylenediamine portion and said bifunctional chelator BFC is DOTA. The anchoring portion A is a functional group meta- or para-bonded to the benzylguanidine of the compound of the invention. The anchoring portion A binds the linker portion L to benzylguanidine. The anchoring portion A can be a chemical group of various nature, and can depend on the linker portion L. Examples of useful anchoring portions A according to the compound of the invention are amide, anilide, ether, amine and sulfonamide groups; preferably the anchoring portion A is an amide group.

In a preferred embodiment, the compound of the invention is the compound of Formula II:

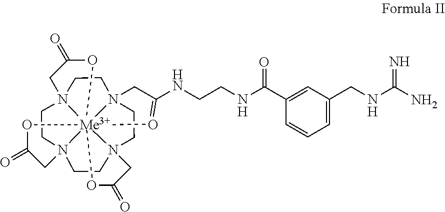

wherein, with reference to the general formula I, Me is the radiation-emitting or non-radiation-emitting metal cation, for example a pure β⁻ radiation-emitting metal cation, such as ¹¹¹Y³⁺. In an alternative embodiment, Me can be a non-radioactive metal cation, for example ⁸⁹Y³⁺, which is useful for the chemical-physical and chemical-biological characterization of the compound of the invention. In further alternative embodiments, Me can be the metal cation ¹⁷⁷Lu³⁺ for teragnostic applications (radio-metabolic therapy exploiting the β⁻ emission and contemporary SPECT imaging exploiting the γ emission), ⁸⁶Y³⁺ and ⁶⁸Ga³⁺ for PET imaging applications PET exploiting the β⁺ emission, and finally ¹¹¹In³⁺ for SPECT imaging applications exploiting they emission. The compound of Formula II is the compound of Formula I wherein the anchoring portion A is a carboxamide group meta-bonded to BG, the linker portion L is an ethylenediamine group and the bifunctional binder BFC is DOTA.

The compound of Formula II has been shown to be particularly effective for its use in tumors overexpressing NET: in fact, the compound of Formula II, wherein Me is ⁸⁹Y³⁺, has been tested on the human neuroblastoma line SK-N-SH in competition experiments with ³H-NE, showing dose-dependent inhibition of the ³H-NE uptake (IC₅₀˜10 μM), without dose-dependent cytotoxic effects evident up to the maximum concentration tested (100 μM).

Additionally, the compound of Formula II, wherein Me is ¹¹¹Y³⁺, has then been prepared with >99% yield of radio-marking and >99% radiochemical purity.

In another embodiment, the compound of the invention is the compound of Formula III:

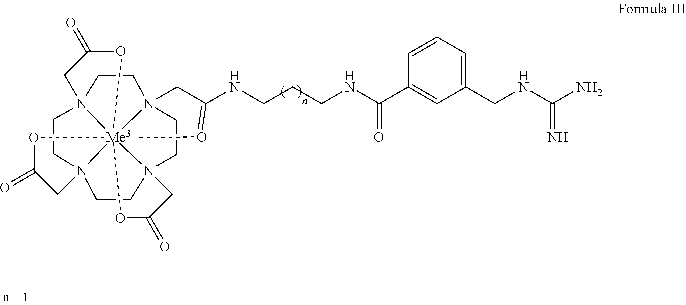

wherein, with reference to formula III, Me is the radiation-emitting or non-radiation-emitting metal cation, for example a pure β⁻ radiation-emitting metal cation, such as ⁹⁰Y³⁺, the anchoring portion A is a carboxamide group meta-bonded to BG, the linker portion L is a propylenediamine group thus containing 3 methylene units, when n is equal to 1, and the bifunctional binder BFC is DOTA.

In an alternative embodiment, Me can be a non-radioactive metal cation, for example ⁸⁹Y³⁺, which is useful for the chemical-physical and chemical-biological characterization of the compound of the invention.

In further alternative embodiments, Me can be the metal cation ¹⁷⁷Lu³⁺ for teragnostic applications (radio-metabolic therapy exploiting the β⁻ emission and contemporary SPECT imaging exploiting the γ emission), ⁸⁶Y³⁺ and ⁶⁸Ga³⁺ for PET imaging applications PET exploiting the β⁺ emission, and finally ¹¹¹In³⁺ for SPECT imaging applications exploiting the γ emission.

According to another embodiment, when n is equal to 2, the compound is of Formula IV.

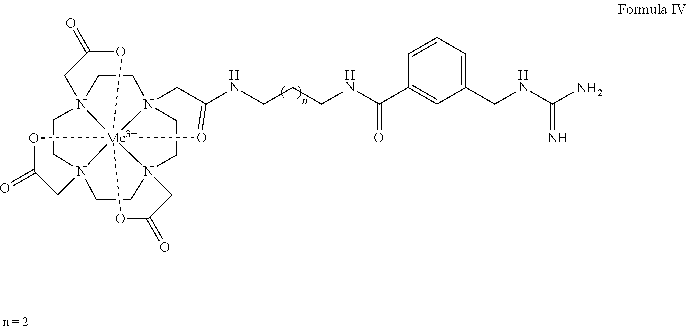

wherein Me is the radiation-emitting or non-radiation-emitting metal cation, for example a pure β⁻ radiation-emitting metal cation, such as ⁹⁰Y³⁺, the anchoring portion A is a carboxamide group meta-bonded to BG, the linker portion L is a butylenediamine group thus containing 4 methylene units, when n is equal to 2, and the bifunctional binder BFC is DOTA.

The compounds of Formula III and IV are two superior homologues of the compound of Formula II, having a linker portion L with 3 and 4 methylene units. The compounds of Formula III and IV according to the invention showed being particularly effective when used in tumors overexpressing NET: in fact, the compounds of Formula III and IV have been tested on the human neuroblastoma line SK-N-SH in competition experiments with ³H-NE, showing a dose-dependent inhibition of the ³H-NE uptake: the compounds of Formula III and IV turned out to be substrates of NET analogously to MIBG and without cytotoxic effects up to the maximum concentration tested (100 μM).

The compound of the invention therefore is a substrate of NET and can have multiple clinical applications, varying for example depending on the type of metal cation Me chosen. The compound of the invention, as it is a pure β⁻ emitter, can in fact find use in RGS of the neuroendocrine tumors overexpressing NET, such as for example pheochromocytomas, paragangliomas, carcinoid tumors and neuroblastomas. Furthermore, the compound of the invention can also be used in the radio-metabolic therapy of the same neuroendocrine tumors, as well as can also find uses in diagnostics, in particular in imaging diagnostics. For example, when Me is the radio-metal ⁶⁸Ga³⁺, the compound of the invention is useful for the PET imaging of the NET-positive neuroendocrine tumors, whereas when Me is ¹¹¹In³⁺, the compound of the invention is useful for the imaging with single-photon emission computed tomography SPECT. Additionally, when Me is ¹⁷⁷Lu³⁺, the compound of the invention has teragnostic function, i.e. therapeutic and diagnostic function, and can be useful in addition to the radio-metabolic therapy also for imaging purposes (SPECT) and dosimetric evaluations.

Therefore, an object of the present invention is also the compound of the invention for its use as a medicament.

An embodiment provides the compound of the invention for its use in the diagnosis and/or treatment of tumors, preferably treatment of neuroendocrine tumors which overexpress the norepinephrine transporter (NET), even more specifically in the treatment of a tumor chosen from the group consisting of pheochromocytoma, paraganglioma, carcinoid tumor and neuroblastoma.

A further embodiment provides the compound of the invention for its use as β⁻ radio-tracer, preferably as pure β⁻ radio-tracer.

Additionally, an embodiment provides the compound of the invention for its use in the radio-guided surgery (RGS). Advantageously, the compound of Formula I, as well as the compounds of Formula II, III and IV, have proven to be particularly effective in the radio-guided surgery by the detection of β⁻ particles (β⁻-RGS). Therefore, an object of the invention is also the use of the compound of the invention in the radio-guided surgery of tumors, preferably by the detection of β⁻ particles (β⁻-RGS).

An object of the present invention is also a treatment method of a subject having a tumor, comprising the administration of an effective dose of the compound of the invention to the subject, wherein the tumor is chosen from neuroendocrine tumors overexpressing NET. Such treatment can provide radio-guided surgery (RGS).

The compounds of the invention can be synthesized by conventional techniques. The general synthesis scheme provides that a benzylguanidine protected at the guanidine group level and suitably functionalized in meta or para position with an anchoring portion, is bonded by conventional coupling methods to a synthon separately prepared which is constituted by BFC suitably protected and previously bonded to the linker portion L. After the coupling, the completely protected conjugate BFC-L-A-BG is subjected to a final deprotection step leading to the free binder, i.e. the compound of Formula I free of Me, ready for the conclusive radio-marking reaction. Such reaction is carried out at a temperature of 85-115° C., preferably 90° C., for a short time period, between 15 and 40 minutes, preferably 30 minutes, in an ammonium acetate buffer at pH=5.5-6 by incubating the binder BFC-L-A-BG with a suitable salt of the radio-metal to be complexed. When the radio-metal is ⁹⁰Y³⁺, the preferred salt is ⁹⁰YCl₃.

The compounds of the invention have an anchoring portion A covalently bonded to the meta or para position of benzylguanidine. Suitably, said anchoring portion A is coupled with the synthon constituted by BFC bonded to the linker portion L, only in the last steps of the synthetic route of the compounds of the invention, thus allowing to have the typical advantages of the “convergent synthetic approaches” such as high synthetic versatility associated with extremely reduced preparation times and costs. Preferably, it is possible to synthesize the compounds of Formula I in the absence of Me, i.e., in some cases, prior to the final radio-marking. This guarantees great versatility in optimizing the pharmacokinetic and pharmacodynamic characteristics of the final chelates, high radiochemical yields/purities and short times, while reducing the problems of radiation protection and saving many radioactivity half-lives of the metal Me to be incorporated which, for the same bifunctional chelator BFC, can be widely varied between different trivalent cations (^{86/90}Y³⁺, ¹¹¹In³⁺, ¹⁷⁷Lu³⁺, etc.), also including radio-metals having short half-life as ⁶⁸Ga³⁺.

An object of the present invention is finally a pharmaceutical composition comprising the compound of the invention and pharmaceutically suitable excipients.

Preferably, such composition is characterized in that the solution, in which the radio-marking is carried out, is diluted with an ammonium acetate solution buffered at pH 7.2-7.4 containing a suitable stabilizer, such as ascorbic acid (the preferred one), gentisic acid or an amino acid solution for infusion, which acting as free radical interceptor inhibits the self-radiolysis of the compound, and after filtration on appropriate filters to ensure their sterility, it is stored in vials ready for intravenous infusion.

### EXPERIMENTAL SECTION

Preparation of the Compound 1 of Formula II

The preparation of the compound 1 of Formula II (also denoted as ⁹⁰Y-DOTA-BG chelate, compound 1 or MC4324) has been carried out as depicted in Scheme 1. The commercial cyclen 2 has been converted to the mono-alkylated intermediate 3 by reaction with ethyl bromoacetate in dichloromethane (DCM) at room temperature. The alkylation reaction of the compound 3 with tert-butyl bromoacetate in the presence of anhydrous potassium carbonate in anhydrous acetonitrile then provided tri-tert-butyl 2,2′,2″-(10-(2-ethoxy-2-oxoethyl)-cyclen-1,4,7-tri-yl)triacetate 4 which has then been treated with an excess of pure ethylenediamine at room temperature to give the synthon A (5) (Scheme 1A). The synthesis of the synthon B (7) instead has been carried out to start from the commercial 3-(aminomethyl)-benzoic acid 6 by guanidination reaction with N,N′-bis-tert-butoxycarbonyl-1H-pyrazol-1-carboxamidine in anhydrous tetrahydrofuran (THF) in the presence of triethylamine (TEA) at room temperature (Scheme 1B). The coupling between the synthon A (5) and the synthon B (7) in the presence of N-(3-dimethylaminopropyl)-N′-ethylcarbodiimide (EDCI), hydrated 1-hydroxybenzotriazole (HOBt) and TEA in anhydrous DCM at room temperature, followed by a total deprotection reaction by using a mixture of trifluoroacetic acid (TFA):tri-isopropylsilane (TIS):water 95/2.5/2.5 (vol/vol/vol) then led to the free binder 9 (MC4325) (Scheme 1C). The DOTA-BG conjugate 9 has then been hot complexed with a slight excess of ⁸⁹Y(NO₃)₃ in the presence of an ammonium acetate buffer at pH 5.5-6 in closed vial to give the non-radioactive analogue, also called “cold tracer”, of the compound of Formula II (1a, MC4324). The DOTA-BG conjugate 9 has been finally used in large excess for the hot radio-marking with ¹¹¹YCl₃ still in the presence of the ammonium acetate buffer at pH 5.5-6 (Scheme 1C) to give the compound of Formula II (1, MC4324).

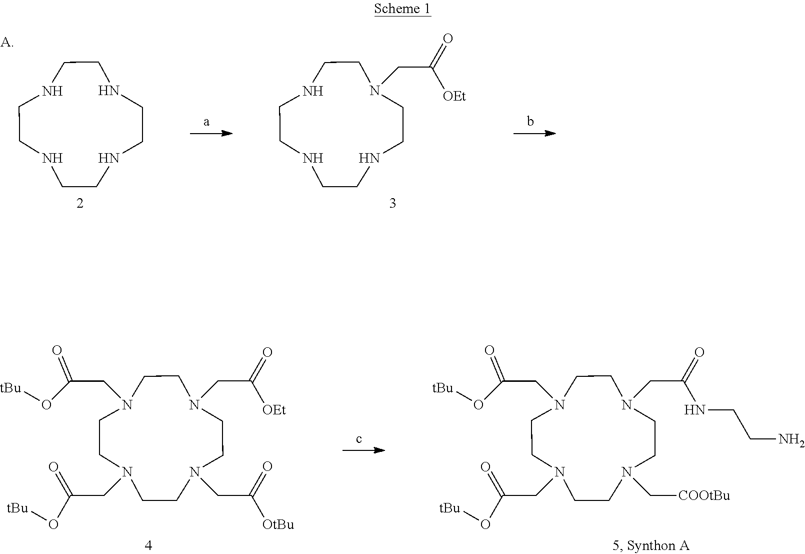

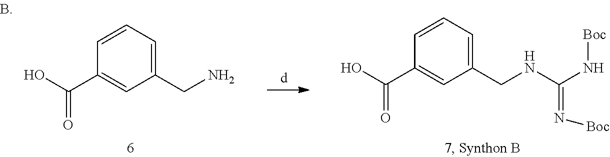 [SMILES: NCc1cccc(C(=O)O)c1 ; CC(C)(C)OC(=O)/N=C(/NCc1cccc(C(=O)O)c1)NC(=O)OC(C)(C)C]

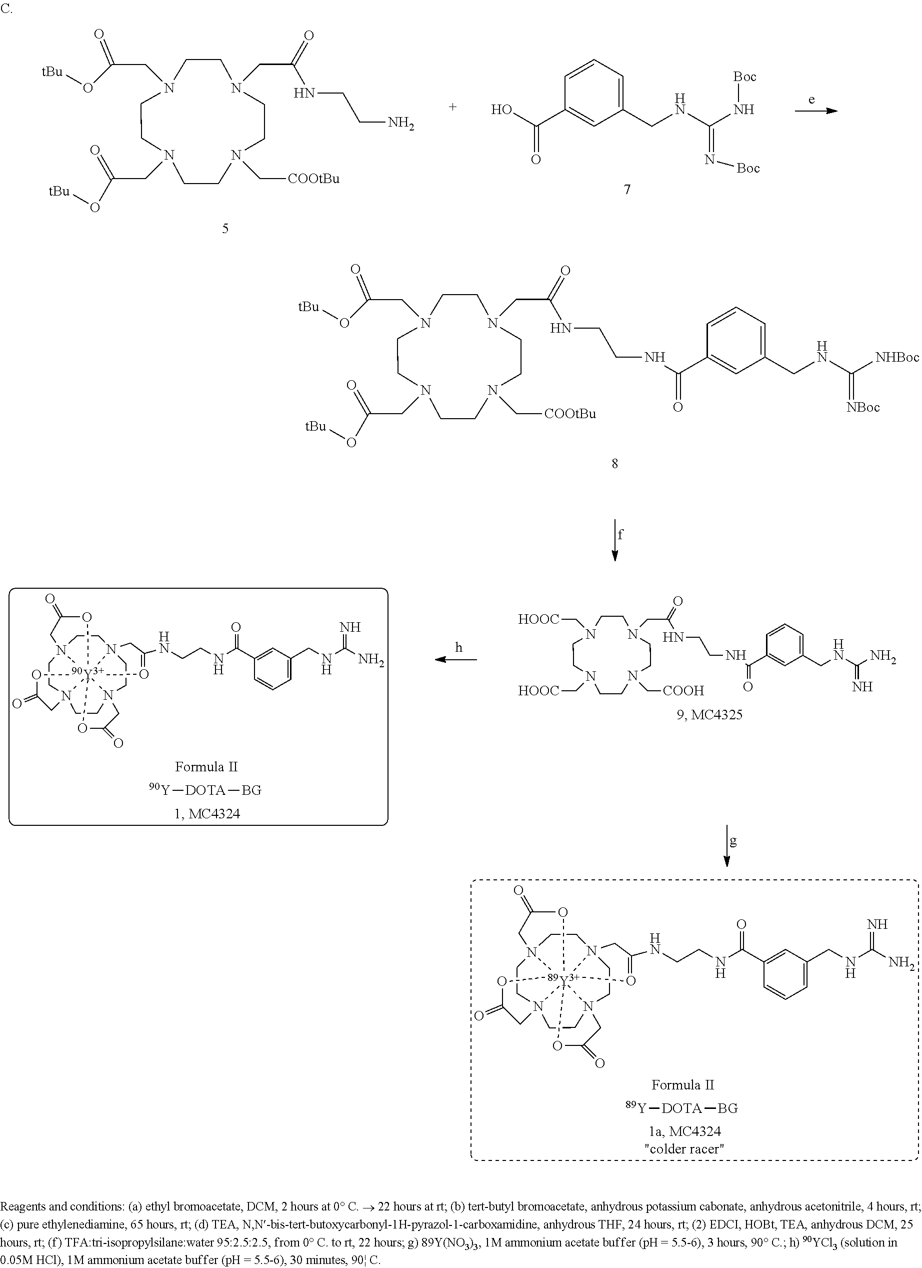

The ¹H-NMR and ¹³C-NMR spectra have been recorded at 400 MHz and 100 MHz, respectively, by using a Bruker AC 400 spectrometer; the chemical shifts are reported in δ units (ppm) with respect to tetramethylsilane used as internal reference (Me₄Si). The ¹H-NMR and ¹³C-NMR spectra of the compound of Formula II (⁸⁹Y-DOTA-BG, compound 1a or MC4324) have been recorded at 600 MHz and 150 MHz, respectively, by using a Bruker AC 600 spectrometer. The low-resolution mass spectra have been recorded on an API-TOF Mariner Perspective Biosystem (Stratford, Tex., USA), the samples have been injected by a Harvard pump by using a flow rate of 5-10 μl/min, infused in the Electrospray system. The high-resolution mass spectra (HR-MS) have been recorded on Orbitrap Exactive spectrometer (Thermo Fisher Scientific, Austin, Tex. USA). The analytical HPLC analyses of the compounds 8, 9 and ⁸⁹Y-DOTA-BG as non-radioactive analogue of the compound of Formula II (1a or MC4324) have been carried out on:
liquid chromatograph UHPLC Accela equipped with pumping system, autosampler and PDA Accela detector coupled to mass spectrometer LTQ ion trap equipped with an ESI interface (Thermo Fisher Scientific, Austin, Tex. USA); the experimental data have been acquired, processed and reworked by Xcalibur software (Thermo Fisher Scientific, Austin, Tex., USA);
Shimadzu Nexera liquid chromatograph equipped with CBM-20A controller, four LC-30AD pumps, a DGU-20 A5R degasser, a SPD-M20A PDA detector (Standard Analytical 2.50 μl Semimicro cell), sampling speed: 100 Hertz; resolution: 4.0 nm), a thermostated column housing CTO-20AC and an autosampler SIL-30AC; an ELSD detector (Sedex 90, SEDERE, France) is connected in series to the system; the experimental data have been acquired, processed and reworked by LabSolution software. (Shimadzu Italia S.r.l., Milan, Italy).

The purification of the compound ⁸⁹Y-DOTA-BG (1a or MC4324) as non-radioactive analogue of the compound of Formula II (1 or MC4324) has been carried out on Waters semi-preparative liquid chromatograph (RP-HPLC) equipped with Waters 590 model pump, 250 μl injector and UV spectrophotometric detector with variable wavelength and Omniscribe recorder on paper.

All the compounds have been regularly controlled by TLC and ¹H-NMR. The TLC has been carried out on silica gel plates supported by aluminum (Merck DC, Alufolien Kieselgel 60 F254) with spots displayed by UV light or by using an alkaline solution of KMnO₄. The concentration of the solutions after the reactions and extractions has been carried out by using a rotary evaporator operating under a reduced pressure of 20 Torr. The organic solutions have been dried on anhydrous sodium anhydrous sulphate or magnesium sulphate. All the chemical reactants have been purchased from Sigma Aldrich s.r.l., Milan (Italy), TCI Europe N.V., Zwijndrecht (Belgium) or Perkin Elmer (USA) and were of the utmost purity. Normally, the samples prepared for physical and biological tests have been dried under high vacuum on P₂O₅ for 20 hours at temperatures between 25 and 40° C., depending on the melting point of the sample. Herein below the experimental procedures for the preparation and chemical-physical characterization of the compounds 1-9 are described.

Synthesis of the compound ethyl 2-(1,4,7,10-tetraazacyclododecan-1-yl)acetate (3). A solution of ethyl bromoacetate (1.0 g; 6.0 mmol) in DCM (10 ml) is added dropwise over 10 minutes to a solution of commercial cyclen 2 (1.36 g, 7.9 mmol) in DCM (16 ml) cooled in an ice bath. After 2 hours, the reaction mixture is brought to room temperature and left to react for additional 22 hours. The resulting suspension is filtered and the filtrate is evaporated under reduced pressure. The crude, oily yellow, reaction product is finally purified by flash chromatography (from 100% DCM to DCM/methanol/ammonia/water 70:30:5:5) thus obtaining the compound 3 (1.61 g, 79%) in form of white viscous oil. ¹H-NMR (CDCl₃) δ ppm: 1.27 (t, J=7.2 Hz, 3H; CH₂CH₃), 2.84 (n, 8H, CH), 2.95 (n, 8H, CH), 3.49 (n, 2H, NCH₂COOCH₂CH₃), 4.16 (q, J=7.2 Hz, 3H, C(O)OCH₂CH₃). MS (ESI): 259 [M+H]+.

Synthesis of tri-tert-butyl 2,2′,2″-(10-(2-ethoxy-2-oxoethyl)-1,4,7,10-tetraazacyclododecane-1,4,7-tri-yl)triacetate (4) To a solution of mono-alkylated cyclen 3 (0.98 g, 3.8 mmoles) in acetonitrile (35 ml), anhydrous potassium carbonate (3.14 g, 22.8 mmoles) is added. To this suspension a solution of tert-butyl bromoacetate (2.22 g, 11.4 mmoles) has been added dropwise in acetonitrile (10 ml) over 20 minutes. The suspension is left under stirring at room temperature for 4 hours. The suspended solid has then been removed by filtration, the solvent evaporated under reduced pressure and the residue has been finally purified by flash chromatography (from 100% DCM to 20% methanol/DCM) to give the compound 4 (1.90 g, 84%) in form of white foam. ¹H-NMR (CDCl₃) δ ppm: 1.15 (t, J=7.2 Hz, 3H, C(O)OCH₂CH₃), 1.33 (s, 9H, tBu), 1.34 (s, 9H, tBu), 1.35 (s, 9H, tBu), 1.80-3.70 (very broad multiplet set with integration corresponding to 24H, CH₂), 4.04 (q, J=7.2 Hz, 3H, C(O)OCH₂CH₃). MS (ESI): 623 [M+Na]+.

Synthesis of tri-tert-butyl 2,2′,2″-(10-(2-((2-aminoethyl)amino)-2-oxoethyl)-1,4,7,10-tetraazacyclododecane-1,4,7-triyl)triacetate (5, Synthon A). The compound 4 (1.31 g, 2.18 mmoles) has been dissolved in pure ethylenediamine (2 ml, 29.9 mmoles) and the resulting solution has been left under stirring at room temperature for 65 hours. At the end of the reaction, ethylenediamine has been removed under reduced pressure and the residue dried under vacuum to give a light yellow foam which finally has been purified by flash chromatography (from 100% DCM to DCM/methanol 1:1) to provide the compound 5 (1.04 g, 72%) in form of white foam. ¹H-NMR (CDCl₃) δ ppm: 1.38 (s, 27H, 3×tBu), 1.48 (m, 2H, NH₂), 2.45 (m, 4H, 2×CH₂), 2.63 (m, 4H, 2×CH₂), 2.72 (t, 2H, CH₂), 2.77-2.80 (m, 8H, 4×CH₂), 2.99 (s, 2H, CH₂), 3.17 (br s, 4H, 2×CH₂), 3.20 (s, 2H, CH₂), 3.21-3.27 (q, 2H, CH₂), 8.68 (t, 1H, CONH); 13C-NMR (CDCl₃) δ ppm: 27.68, 27.84, 36.60, 40.03, 55.39, 55.51, 55.90, 81.61, 81.70, 172.17, 172.27. MS (ESI): 637 [M+Na]+.

Synthesis of N,N′-bis-tert-butoxycarbonyl-3-guanidinomethyl-benzoic acid (7, Synthon B). To a suspension of commercial 3-(aminomethyl)-benzoic acid 6 (1.66 mmol, 250.0 mg) in a mixture of methanol anhydrous (16.5 ml) and anhydrous THF (16.5 ml), TEA (6.61 mmol, 669.4 mg, 0.92 ml) and N,N-bis-tert-butoxycarbonyl-1H-pyrazole-1-carboxamidine (1.98 mmol, 615.9 mg) have been sequentially added and the resulting mixture has been left under stirring for 24 hours at room temperature. At the completion of the reaction, the solvent has been evaporated under reduced pressure and the residue has been dissolved in ethyl acetate and washed in succession with 0.1 N potassium bisulphate and sodium chloride saturated solution. Thus, the organic phase has been dried over magnesium sulphate and the solvent removed under reduced pressure. The crude reaction product has been finally purified by flash chromatography on silica gel (Biotage Isolera One®) by eluting with a chloroform/methanol 0→6% mixture to provide the intermediate 7 as a white solid. ¹H-NMR (DMSO-d6) δ ppm: 1.38 (s, 9H, C(CH3)3), 1.49 (s, 9H, C(CH₃)₃), 4.58 (d, 2H, NHCH₂Ph-COOH), 7.45-7.51 (t, 1H, CH benzene ring), 7.54-7.56 (m, 1H, CH benzene ring), 7.83-7.85 (m, 1H, CH benzene ring), 7.89 (s, 1H, CH benzene ring), 8.76-8.79 (t, 1H, NHCH₂Ph-COOH), 11.53 (s, 1H, Boc-NH), 12.98 (br s, 1H, COOH). MS (ESI): 392 [M−H]−.

Synthesis of tri-tert-butyl 2,2′,2″-(10-(2-((2-(3-((2,3-bis(tert-butoxycarbonyl)guanidino) methyl)benzamido)ethyl)amino)-2-oxoethyl)-1,4,7,10-tetraazacyclododecan-1,4,7-triyl) triacetate (8). To a solution of N,N-bis-tert-butoxycarbonyl-3-guanidinomethyl-benzoic acid 7 (0.16 mmol, 64.0 mg) in anhydrous DCM (1.5 ml), HOBt (0.21 mmol, 28.6 mg), EDCI (0.21 mmol, 40.5 mg), tri-tert-butyl 2,2′,2″-(10-(2-((2-aminoethyl)amino)-2-oxoethyl)-cyclen-1,4,7-triyl)triacetate 5 (100.0 mg, 0.16 mmol) and finally TEA (0.58 mmol, 59.2 mg, 81.6 μl) are added in succession by cooling in ice bath. After about 25 hours at room temperature, the solvent is removed by evaporation and the resulting crude product has been purified by flash chromatography on silica gel (Biotage Isolera One®) by eluting with a chloroform/methanol 0→8% mixture to provide the compound 8 (70.5 mg, yield=44%) in form of white solid. ¹H-NMR (CDCl₃) δ ppm: 1.35 (s, 9H, tBu), 1.39 (s, 27H, 3×tBu), 1.44 (s, 9H, tBu), 1.85-3.65 (very broad multiplet set with integration corresponding to 28H, 14×CH₂), 4.60 (d, 2H, NHCH₂), 7.33-7.36 (in, 2H, CH benzene ring), 7.99 (s, 1H, CH benzene ring), 8.15 (d, 1H, CH benzene ring), 8.49 (m, 1H, NHCH₂), 8.81 (m, 1H, CONH), 9.03 (m, 1H, CONH), 11.5 (s, 1H, BocNH). MS (ESI): 990 [M+H]+.

Synthesis of 2,2′,2″-(10-(2-((2-(3-(guanidinomethyl)benzamido)ethyl)amino)-2-oxoethyl)-1,4,7,10-tetraazacyclododecane-1,4,7-triyl) triacetic acid (9, also denoted as MC4325 or DOTA-BG conjugate) A mixture of TFA: tri-isopropylsilane:water 95:2.5:2.5 v:v:v (6.0 ml) has been added to the intermediate 8 (0.091 mmol, 90.0 mg) by cooling at 0° C. The deprotection reaction has been checked by RP-HPLC in the following analytical conditions: column: Sunfire C18, 3.5 μm (150*4.6 mm ID); Eluents: A) H2O/ACN 95/5+0.1% TFA, B) ACN+0.1% TFA. Gradient elution (start A/B 100/0; 1 min A/B 100/0; 15 min A/B 0/100, 20 min A/B 0/100). Flow: 1.0 ml/min. Detection: PDA (200-400 nm), ELSD (T: 80° C., P: 4 bar), T col=30° C.; samples dissolved in MeOH. After about 22 hours at room temperature the reaction proved to be completed. Thus, the solvent has been evaporated and the residue co-evaporated several times with anhydrous DCM (6×3 ml) and anhydrous diethyl ether (5×3 ml). The crude product thus obtained has then been triturated with anhydrous diethyl ether, filtered and washed on filter with anhydrous diethyl ether to provide the desired compound 9 (88.2 mg, yield=81%) in form of white solid. ¹H-NMR (D₂O) δ ppm: 2.90-3.75 (very broad multiplet set with integration corresponding to 28H, 14×CH₂), 4.41 (d, 2H, NHCH₂), 7.43 (m, 2H, CH benzene ring), 7.62 (m, 2H, CH benzene ring).

HR-MS (ESI): calculated mass for [C₂₇H₄₃N₉O₈+H]⁺=622.3307 m/z; determined mass: 622.3294 m/z. Source parameters: capillary temperature 275° C.; spray voltage 3.70 kV; sheath gas 6; tube lens voltage 125 V. Resolution input: 100000. Samples dissolved in MeOH (10⁰M).

Preparation of the chelate ⁸⁹Y-DOTA-BG as non-radioactive analogue of the compound of Formula II (1a, MC4324). To the binder 9 (36.0 mg, 30.2 μmol) dissolved in 1 ml of 1M ammonium acetate buffer (pH=5.5-6), the commercial salt 89Y(NO₃)₃.4H₂O (15.7 mg, 45.3 μmol) has been added. The resulting solution has then been hot stirred (90° C.) in closed vial for 3 hours. At the end of the reaction, the mixture has been left to cool at room temperature and then the crude chelate has been purified from the excess salts by semi-preparative HPLC (conditions: column Hypersil ODS GOLD 5 μm; 250×10 mm; eluent: MeOH/H₂O 5:95 v/v+0.02% TFA, flow: 4.0 ml/min; UV detection 254 nm) until providing, after lyophilization of the clean fractions, the “cold” chelate 89Y-DOTA-BG 1a (MC4324) as a white powdered solid (19 mg, yield=90%). ¹H-NMR (D₂O, 600 MHz, T=5° C.) δ ppm: 2.08 (d, 1H, CHH), 2.22-2.33 (m, 5H, 2×CH, and CHH), 2.53-2.67 (m, 7H, 3×CH, and CHH), 2.98 (d, 1H, CH), 3.08 (d, 2H, CH₂), 3.18-3.40 (m, 6H, 3×CH₂), 3.43-3.56 (m, 4H, 2×CH₂), 3.64 (m, 2H, CH₂), 4.38 (d, 2H, —CH₂-guanidine), 7.43 (m, 2H, CH benzene ring), 7.60 (m, 2H, CH benzene ring). 13C-NMR (D₂O, 150 MHz, T=5° C.) δ ppm: 180.29, 179.71, 175.80, 169.98, 156.53, 136.86, 133.23, 130.63, 129.15, 129.13, 126.19, 125.21, 65.46, 65.32, 65.22, 63.04, 55.64, 55.47, 55.46, 55.35, 54.73, 54.72, 54.59, 54.18, 43.75, 39.20, 38.40.

HR-MS (ESI): calculated mass for [C₂₇H₄₀N₉O₈Y+H]⁺=708.2131 m/z; determined mass: 708.2124 m/z. Source parameters: capillary temperature 275° C.; spray voltage 3.70 kV; sheath gas 6; tube lens voltage 125 V. Resolution input: 100000. Samples dissolved in MeOH (10⁻⁵M).

Preparation of the compound of Formula II (1, ⁹⁰Y-DOTA-BG, MC4324). The chelate ⁹⁰Y-DOTA-BG of Formula II (1) has been prepared by adding 30 μl of a commercial solution of 90YCl3 in 0.05 M HCl (1.07 mCi, 22.1 μmol) to 132 μl of a 0.1 mM binder 9 solution (13.2 nmol) in 1M ammonium acetate buffer (pH=5.5-6) and bringing to the final volume of 500 μl by the addition of 338 μl of the same 1M ammonium acetate buffer (pH=5.5-6). The resulting solution has then been incubated in closed vial placed on a heating block under shielded hood at 90° C. for 30 minutes. At the end of the reaction, after cooling to room temperature, different aliquots of the solution (2-10 μl) have been collected without dilution to evaluate the radio-marking yield and to carry out the quality control by ITLC. The ITLC methods used ITLC-SG and ITLC-SA Agilent plates and different eluent systems such as 1M ammonium acetate buffer (pH=5.5-6):methanol (50:50 v/v), 1M ammonium acetate buffer (pH=5.5-6):methanol:ammonia 33% (50:50:5 v/v/v) and 50 mM EDTA in 0.1 M ammonium acetate buffer (pH=6). The results of different chromatographic runs showed in agreement >99% radio-marking yield and radiochemical purity (FIG. 1). FIG. 1 depicts the ITLC-SG runs relative to the radio-marking of the binder 9 with ⁹⁰YCl_{b} for the preparation of the chelate ⁹⁰Y-DOTA-BG of Formula II (1, or MC4324).

Chemical Stability of the Chelate ⁸⁹Y-DOTA-BG (1a, MC4324) as Non-Radioactive Analogue of the Compound of Formula II (1, MC4324) in Physiological Conditions.

The stability of the chelate ⁸⁹Y-DOTA-BG (1a, MC4324) (non-radioactive analogue of the compound of Formula II, 1 or MC4324) in physiological conditions has been evaluated by analysis with analytical HPLC (Shimadzu Nexera chromatograph equipped with a SPD-M20A PDA detector; Hypersil ODS GOLD 250×4.6 mm column; eluent: MeOH/H₂O 5:95 v/v+0.02% TFA, flow: 1.0 ml/min; UV detection 214 nm) repeated at regular time intervals after having solubilized it in PBS buffer (c=0.9 mg/ml) and kept at T=37° C. After 5 days, the chelate was found to be still perfectly intact, with a chemical purity constantly higher than 99.5% (see Table 1). The chelate ⁸⁹Y-DOTA-BG (peak 1 in the chromatogram in FIG. 2A-C) immediately revealed the presence of a peak 2 (lower than 0.1%) which has also remained constant over time.

FIG. 2 depicts the HPLC plot relative to the compound ⁸⁹Y-DOTA-BG (1a) solubilized in PBS buffer (c=0.9 mg/ml) and at T=37° C. time t=0 min (A), t=21 hours (B) and t=5 days (C).

TABLE 1
Trend over time of the areas of the chromatographic peaks
relative to the compound 1a ⁸⁹Y-DOTA-BG (1) and impurity 2.

| | | |
|---|---|---|
| Time | Area 1 | Area (1 + 2) |

| | | | |
|---|---|---|---|
| 0 | min | 34.78 | 35.13 |
| 30 | min | 35.09 | 35.29 |

| | | |
|---|---|---|
| 1 hour 30 min | 35.07 | 35.30 |

| | | | |
|---|---|---|---|
| 5 | hours | 35.13 | 35.40 |
| 21 | hours | 37.15 | 37.45 |
| 5 | days | 38.34 | 38.65 |

Biological Validation of the ⁸⁹Y-DOTA-BG Chelate (1a, MC4324) (Non-Radioactive Analogue of the Compound of Formula II, 1 or MC4324) as Substrate of NET in the Human Neuroblastoma Line SK-N-SH.

The ability of the ⁸⁹Y-DOTA-BG chelate (1a, MC4324) (non-radioactive analogue of the compound of Formula II, 1 or MC4324) to act as substrate of NET has been evaluated in competition experiments with the tritiated endogenous substrate ³H-NE for the uptake by the human neuroblastoma cells SK-N-SH, which are known to express the transporter NET in large quantity. In this cell line, the ⁸⁹Y-DOTA-BG chelate showed dose-dependent inhibition of the uptake/internalization of ³H-NE (IC₅₀˜10 μM), showing to be a substrate of NET analogously to MIBG, even though with lower power (FIG. 3) and without dose-dependent cytotoxic effects which are evident up to the maximum concentration tested (100 μM). It is interesting to note how the analogous of the ⁸⁹Y-DOTA-BG chelate not containing yttrium, the free binder 9 (MC4325), was completely unable to compete with ³H-NE for the internalization (FIG. 3). This provides the ability to prepare radio-marked versions of the chelate of Formula II (or MC4324) with an even low specific activity, because the absence of competition for NET between the chelate (MC4324) and the free binder 9 (MC4325) doesn't compromise the bonding ability to NET of the chelate (compound of Formula II, MC4324) also in the presence of an excess of free binder 9 (MC4325).

FIG. 3 depicts the competition of the ⁸⁹Y-DOTA-BG chelate (1a, MC4324) (non-radioactive analogue of the compound of Formula II, 1 or MC4324) and of free binder 9 (MC4325) with ³H-NE (50 nM, 1 hour incubation, T=37° C.) for the uptake by the SK-N-SH cells. ¹H-NE a 20 μM and MIBG @ 2 μM have been used as positive controls.

Materials and Methods Relative to the Cell Tests

Cell Lines and Culture Conditions

The human neuroblastoma cell line SK-N-SH has been purchased from ATCC. The cells have been kept in E-MEM medium containing 10% fetal bovine serum (FBS), 2 mM L-glutamine and antibiotics in humidified atmosphere with 5% CO₂ at 37° C.

Cell Uptake of ³H-NE

³H-NE has been purchased from Perkin Elmer. To measure the initial cell uptake speeds of ³H-NE, the cells have been grown for 16 hours in the presence of serum only, then incubated with binder medium (EMEM containing 0.2% BSA and 20 mM Hepes, pH 7.5) for 10 minutes by heating in a water bath at 37° C. The bond has been initiated by adding 0.5 ml per well of 50 nM ³H-NE to the binder medium for a long time, the plates have then been placed on ice and washed three times with frozen PBS. Thus, the cell monolayers have been dried and lysed with 2% NaOH 1N SDS. The cpm (“counts” per minute) have been determined separately in the different wells in triple for each time. By cpm is meant “counts” or “hits” per minute, i.e. the nuclear disintegrations that occur in one minute. These disintegrations come from inside the cells after these have been, following the order below: 1) incubated with ³H-NE, 2) washed with PBS to eliminate ³H-NE remained outside the cells, 3) lysed for better detecting the radioactivity due to the internalized ³H-NE only. The cpm are thus due to ³H-NE which is internalized by the cell and are maximum when no competition occurs (control/non-treated or better treated cells with ³H-NE only) whereas cpm decrease in a more marked way the more effective the competition with ³H-NE of the substance tested each time is.

The background bond has been determined in parallel in a fourth well containing a 400 times molar excess of non-marked ¹H-NE (“cold”). For the competition study, the cells have been plated as above, and thus incubated for 1 hour with 50 nM ³H-NE in binder medium in the absence and presence of the ⁸⁹Y-DOTA-BG chelate (compound of Formula II, 1a or MC4324) (non-radioactive analogue of the compound of Formula II, 1 or MC4324) and free binder 9 (MC4325) at different concentrations. The radioactivity has been determined separately in the wells in triple for each concentration value. The uptake of ³H-NE has been expressed as total radioactivity percentage normalized for mg of protein.

Stability in Serum of the Chelate ⁹⁰Y-DOTA-BG of Formula II (1 or MC4324).

The stability in serum of the ¹¹¹Y-DOTA-BG chelate of Formula II (1 or MC4324) has been evaluated by measuring the release of the metal cation ⁹⁰Y³⁺ from the chelate to the serum proteins during 14 days in which it has been kept in physiological conditions. Briefly, the ⁹⁰Y-DOTA-BG chelate of Formula II (1 or MC4324) has been incubated with human serum (32 MBq for 16 ml of serum) at T=37° C. At regular time intervals serum aliquots have been collected and, by using centrifuge filter tubes (Amicon® Ultra-4 3K, Merck Millipore) and by centrifuging at 5500 g, the serum proteins have been separated from the non-protein fraction of serum and the radioactivity of both fractions has been measured with a liquid scintillation β counter (scintillation liquid used is Perkin Elmer ULTIMA GOLD). During the 14 days of observation no measurable loss of radioactivity (⁹⁰Y³⁺) in favor of the protein fraction of serum has been detected.

Preparation of the Compounds of Formula III and IV

The preparation of the compounds of Formula III and IV (also denoted as compounds 10a, 10a′ and 10b, 10b′ or MC4801 and MC4803, respectively) has been carried out as depicted in Scheme 2.

In particular, the compounds 10a and 10a′ correspond to the general formula III when n is equal to 1, and Me is chosen from ⁹⁰Y³⁺ or ⁸⁹Y³⁺, respectively. The compounds 10b and 10b′ correspond instead to the general formula IV when n is equal to 2, and Me is chosen from ⁸⁹Y³⁺ or ⁸⁹Y³⁺, respectively.

Tri-tert-butyl 2,2′,2″-(10-(2-ethoxy-2-oxoethyl)-cyclen-1,4,7-tri-yl)triacetate 4, prepared as shown in Scheme 1, has been treated with an excess of the suitable commercial diamine (1,3-propanediamine or 1,4-butanediamine) at room temperature to give the respective intermediates of the synthon A′ 11a and 11b. These have then been conjugated with the synthon B (7) prepared as reported in Scheme 1, in the presence of N-(3-dimethylaminopropyl)-N′-ethylcarbodiimide (EDCI), hydrated 1-hydroxybenzotriazole (HOBt) and TEA in anhydrous DCM at room temperature to give the completely protected derivatives 12a and 12b. These have then been subjected to total deprotection reaction by using a trifluoroacetic acid (TFA):tri-isopropylsilane (TIS):water 95/2.5/2.5 (vol/vol/vol) mixture leading to the respective free binders 13a (MC4802) and 13b (MC4804). Thus the free binders 13a,b have been hot complexed with a slight excess of ⁸⁹Y(NO₃); in the presence of an ammonium acetate buffer at pH 5.5-6 in closed vial to give the non-radioactive analogues, also called “cold tracers”, of the compounds of Formula III (10a′, MC4801) and IV (10b′, MC4803). The free binders 13a,b have finally been used in large excess for the hot radio-marking with ⁹⁰YCl₃ still in the presence of the ammonium acetate buffer at pH 5.5-6, to give the compounds of Formula III (10a, MC4801) and IV (10b, MC4803).

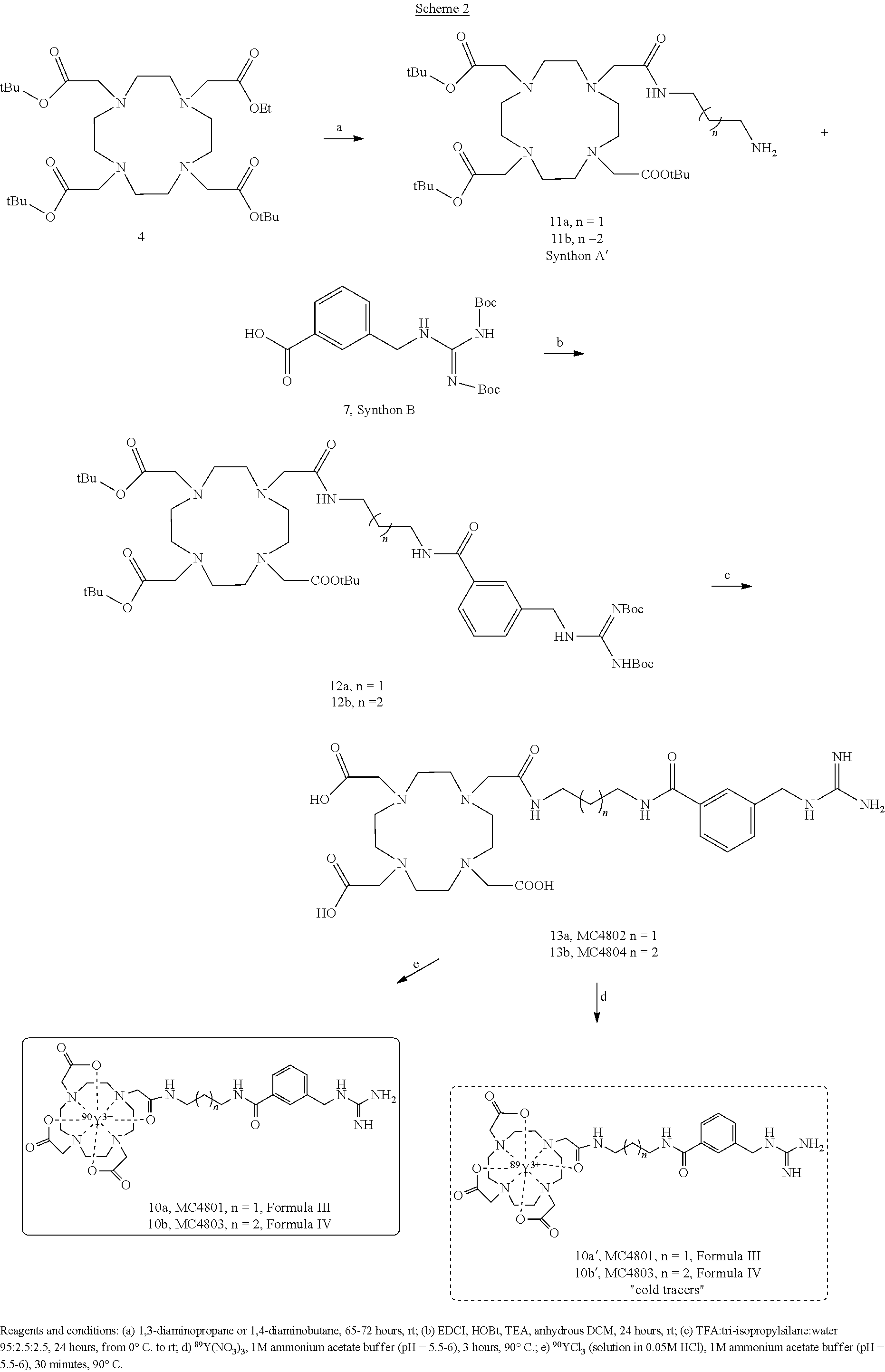

The ¹H-NMR spectra of synthetic intermediates 11-13 have been recorded at 400 MHz by using a Bruker AC 400 spectrometer, whereas the ¹H-NMR and ¹³C-NMR spectra of the compounds of Formula III and IV 10a′ (MC4801) and 10b′ (MC4803) have been recorded at 600 MHz and 150 MHz, respectively, by using a Bruker AC 600 spectrometer; the chemical shifts are reported in δ units (ppm) with respect to tetramethylsilane used as internal reference (Me₄Si). The low-resolution mass spectra have been recorded on an API-TOF Mariner Perspective Biosystem (Stratford, Tex., USA), the samples have been injected by a Harvard pump by using a flow rate of 5-10 μl/min, infused in the Electrospray system. The high-resolution mass spectra (HR-MS) have been recorded on Orbitrap Exactive spectrometer (Thermo Fisher Scientific, Austin, Tex. USA). The analytical HPLC analyses of the compounds 12a,b, and 13a,b and compounds 10a′,b′ as non-radioactive analogues of the compounds of Formula III (10a, MC4801) and IV (10b, MC4803) have been carried out on:
liquid chromatograph UHPLC Accela equipped with pumping system, autosampler and PDA Accela detector coupled to mass spectrometer LTQ ion trap equipped with an ESI interface (Thermo Fisher Scientific, Austin, Tex. USA); the experimental data have been acquired, processed and reworked by Xcalibur software (Thermo Fisher Scientific, Austin, Tex., USA);
Shimadzu Nexera liquid chromatograph equipped with CBM-20A controller, four LC-30AD pumps, a DGU-20 A5R degasser, a SPD-M20A PDA detector (Standard Analytical 2.50 μl Semimicro cell), sampling speed: 100 Hertz; resolution: 4.0 nm), a thermostated column housing CTO-20AC and an autosampler SIL-30AC; an ELSD detector (Sedex 90, SEDERE, France) is connected in series to the system; the experimental data have been acquired, processed and reworked by LabSolution software. (Shimadzu Italia S.r.l., Milan, Italy).

The purification of the compounds containing ⁸⁹Y³⁺ [compounds of Formula III (10a′, MC4801) and IV (10b′, MC4803)] as non-radioactive analogues of the compounds of Formula III (10a, MC4801) and IV (10b, MC4803) has been carried out on Waters semi-preparative liquid chromatograph (RP-HPLC) equipped with Waters 590 model pump, 250 μl injector and UV spectrophotometric detector with variable wavelength and Omniscribe recorder on paper.

All the compounds have been regularly controlled by TLC and ¹H-NMR. The TLC has been carried out on silica gel plates supported by aluminum (Merck DC, Alufolien Kieselgel 60 F254) with spots displayed by UV light or by using an alkaline solution of KMnO₄. The concentration of the solutions after the reactions and extractions has been carried out by using a rotary evaporator operating under a reduced pressure of 20 Torr. The organic solutions have been dried on anhydrous sodium anhydrous sulphate or magnesium sulphate. All the chemical reagents have been purchased from Sigma Aldrich s.r.l., Milan (Italy), TCI Europe N.V., Zwijndrecht (Belgium) or Perkin Elmer (USA) and were of the utmost purity. Normally, the samples prepared for physical and biological tests have been dried under high vacuum on P₂O₅ for 20 hours at temperatures between 25 and 40° C., depending on the melting point of the sample.

Herein below the experimental procedures for the preparation and chemical-physical characterization of the compounds 10-13 are described.

General procedure for the preparation of the intermediates 11a,b. Example: tri-tert-butyl 2,2′,2″-(10-(2-((3-aminopropyl)amino)-2-oxoethyl)-1,4,7,10-tetraazacyclododecan-1,4,7-triyl)triacetate (11a). The compound 4 (1.0 g, 1.66 mmoles) has been dissolved in commercial, pure 1,3-diamino propane (1.5 ml, 17.9 mmoles) and the resulting solution has been left under stirring at room temperature for 72 hours. At the end of the reaction, the propylenediamine has been removed under reduced pressure and the residue dried under vacuum to give a dark yellow foam which finally has been purified by flash chromatography (from 100% DCM to DCM/methanol 40:60 v/v) to provide the compound 11a (0.78 g, 75%) in form of white foam. ¹H-NMR (CDCl3) δ ppm: 1.39 (s, 27H, 3×tBu), 1.47 (m, 2H, NH₂), 1.85 (n, 2H, CONHCH₂CH₂CH₂NH), 2.44 (m, 4H, 2×CH₂), 2.63 (m, 4H, 2×CH₂), 2.66 (m, 2H, CONHCH₂CH₂CH₂NH₂), 2.76-2.81 (m, 8H, 4×CH₂), 2.98 (s, 2H, NCH₂CONH), 3.16 (br s, 4H, 2×NCH₂COO/Bu), 3.20 (s, 2H, NCH₂COOtBu), 3.15-3.18 (m, 2H, COHCH₂CH₂CH₂NH₂), 8.68 (t, 1H, CONH); MS (ESI): 629 [M+H]⁺.

2,2′,2″-(10-(2-((4-aminobutyl)amino)-2-oxoethyl)-1,4,7,10-tetraazacyclododecan-1,4,7-triyl)triacetate (11b). ¹H-NMR (CDCl₃) δ ppm: 1.38 (s, 27H, 3×tBu), 1.48 (m, 2H, NH₂), 1.51 (m, 4H, CONHCH₂CH₂CH₂CH₂NH₂), 2.45 (m, 4H, 2×CH₂), 2.63 (m, 4H, 2×CH₂), 2.67 (m, 2H, CONHCH₂CH₂CH₂CH₂NH), 2.77-2.82 (m, 8H, 4×CH₂), 2.99 (s, 2H, NCH₂CONH), 3.02-3.05 (m, 2H, CONHCH₂CH₂CH₂CH₂NH₂), 3.17 (br s, 4H, 2×NCH₂COO/Bu), 3.21 (s, 2H, NCH₂COO/Bu), 8.67 (t, 1H, CONH); MS (ESI): 643 [M+H]⁺.

General procedure for the preparation of the intermediates 12a,b. Example: tri-tert-butyl 2,2′,2″-(10-(2-((4-(3-((2,3-bis(tert butoxycarbonyl)guanidino)methyl)benzamido)butyl)amino)-2-oxoethyl)-1,4,7,10-tetraazacyclododecan-1,4,7-triyl)(Z)-triacetate (12b).

To a solution of N,N′-bis-tert-butoxycarbonyl-3-guanidinomethyl-benzoic acid 7 (0.16 mmol, 65.0 mg) in anhydrous DCM (1.5 ml), HOBt (0.21 mmol, 29.0 mg), EDCI (0.21 mmol, 41.0 mg), tri-tert-butyl 2,2′,2″-(10-(2-((4-aminobutyl)amino)-2-oxoethyl)-1,4,7,10-tetraazacyclododecan-1,4,7-triyl)triacetate 11b (102.9 mg, 0.16 mmol) and finally TEA (0.58 mmol, 59.2 mg, 82.0 μl) are added in succession by cooling in ice batch. After about 24 hours at room temperature, the solvent is removed by evaporation and the resulting crude product has been purified by flash chromatography on silica gel (Biotage Isolera One®) by eluting with a chloroform/methanol 0→9% mixture to provide the compound 12b (84.7 mg, yield=52%) in form of white solid. ¹H-NMR (CDCl₃) δ ppm: 1.35 (s, 9H, tBu), 1.39 (s, 27H, 3×tBu), 1.44 (s, 9H, tBu), 1.49-1.57 (m, 4H, CONHCH₂CH₂CH₂CH₂NHCO), 1.92-3.58 (very broad multiplet set with integration corresponding to 28H, 14×CH₂), 4.63 (d, 2H, NHCH₂), 7.34-7.38 (m, 2H, CH benzene ring), 7.95 (m, 1H, CH benzene ring), 8.12 (d, 1H, CH benzene ring), 8.49 (m, 1H, NHCH₂), 8.81 (m, 1H, CH₂CONH), 9.03 (m, 1H, PhCONH), 11.4 (s, 1H, BocNH). MS (ESI): 1019 [M+H]⁺.

Tri-tert-butyl 2,2′,2″-(10-(2-((3-(3-((2,3-bis(tert-butoxycarbonyl)guanidino)methyl)benzamido)propyl)amino)-2-oxoethyl)-1,4,7,10-tetraaza cyclododecan-1,4,7-triyl)(Z)-triacetate (12a). ¹H-NMR (CDCl₃) δ ppm: 1.35 (s, 9H, tBu), 1.39 (s, 27H, 3×tBu), 1.44 (s, 9H, tBu), 1.70-1.76 (m, 2H, CONCH₂CH₂CH₂NHCO), 1.92-3.58 (very broad multiplet set with integration corresponding to 28H, 14×CH₂), 4.63-4.65 (d, 2H, NHCH₂), 7.35-7.39 (m, 2H, CH benzene ring), 7.97 (m, 1H, CH benzene ring), 8.16 (d, 1H, CH benzene ring), 8.51 (m, 1H, NHCH₂), 8.83 (m, 1H, CH CONH), 9.07 (m, 1H, PhCONH), 11.6 (s, 1H, BocNH). MS (ESI): 1005 [M+H]+.

General procedure for the preparation of the intermediates 13a,b. Example: 2,2′,2″-(10-(2-((3-(3-(guanidinomethyl)benzamido)propyl)amino)-2-oxoethyl)-1,4,7,10-tetraazacyclododecane-1,4,7-triyl) triacetic acid (13a, also denoted as MC4802). A mixture of TFA:tri-isopropylsilane:water 95:2.5:2.5 v:v:v (6.0 ml) has been added to the intermediate 12a (0.099 mmol, 100.0 mg) by cooling at 0° C. The deprotection reaction has been checked by RP-HPLC in the following analytical conditions: column: Sunfire C18, 3.5 μm (150*4.6 mm ID); Eluents: A) H2O/ACN 95/5+0.1% TFA, B) ACN+0.1% TFA. Gradient elution (start A/B 100/0; 1 min A/B 100/0; 15 min A/B 0/100, 20 min A/B 0/100). Flow: 1.0 ml/min. Detection: PDA (200-400 nm), ELSD (T: 80° C., P: 4 bar); T col=30° C.; samples dissolved in MeOH. After about 24 hours at room temperature the reaction proved to be completed. Thus, the solvent has been evaporated and the residue co-evaporated several times with anhydrous DCM (6×4 ml) and anhydrous diethyl ether (6×3 ml). The thus obtained crude product has then been triturated with anhydrous diethyl ether, filtered and washed on filter with anhydrous diethyl ether to provide the desired compound 13a (53.4 mg, yield=85%) in form of white solid. ¹H-NMR (D2O) δ ppm: 1.70-1.76 (m, 2H, CONHCH₂CH₂CH₂NHCO), 2.92-3.73 (very broad multiplet set with integration corresponding to 28H, 14×CH₂), 4.42 (d, 2H, NHCH₂), 7.45 (m, 2H, CH benzene ring), 7.63 (m, 2H, CH benzene ring).

HR-MS (ESI): calculated mass for [C₂₈H₄₅N₉O₈+H]⁺=636.3469 m/z; determined mass: 636.3456 m/z. Source parameters: capillary temperature 275° C.; spray voltage 3.70 kV; sheath gas 6; tube lens voltage 125 V. Resolution input: 100000. Samples dissolved in MeOH (10⁻⁵ M).

2,2′,2″-(10-(2-((3-(3-(guanidinomethyl)benzamido)butyl)amino)-2-oxoethyl)-1,4,7,10-tetraazacyclododecane-1,4,7-triyl) Triacetic Acid (13b, also Denoted as MC4804).

¹H-NMR (D₂O) δ ppm: 1.50-1.61 (m 4H, CONHCH₂CH₂CH₂CH₂NHCO), 2.88-3.77 (very broad multiplet set with integration corresponding to 28H, 14×CH₂), 4.39 (d, 2H, NHCH₂), 7.44-7.47 (m, 2H, CH benzene ring), 7.60-7.63 (m, 2H, CH benzene ring).

HR-MS (ESI): calculated mass for [C₂₉H₄₇N₉O₈+H]⁺=650.3626 m/z; determined mass: 650.3612 m/z. Source parameters: capillary temperature 275° C.; spray voltage 3.70 kV; sheath gas 6; tube lens voltage 125 V. Resolution input: 100000. Samples dissolved in MeOH (10⁻⁵ M).

General procedure for the preparation of the chelates of Formula III (10a′, MC4801) and IV (10b′, MC4803) in the “cold” versions. Example: chelate 10a′ (MC4801). To the binder 13a (19.1 mg, 30.2 μmol) dissolved in 1 ml of 1M ammonium acetate buffer (pH=5.5-6), the commercial salt 89Y(NO3)3.4H2O (15.7 mg, 45.3 μmol) has been added. The resulting solution has then been hot stirred (90° C.) in closed vial for 3 hours. At the end of the reaction, the mixture has been left to cool at room temperature and then the crude chelate has been purified from the excess salts by semi-preparative HPLC (conditions: column Hypersil ODS GOLD 5 μm: 250×10 mm; eluent: MeOH/H2O 5:95 v/v+0.02% TFA, flow: 4.0 ml/min; UV detection 254 nm) until providing, after lyophilization of the clean fractions, the “cold” chelate 10a′ (MC4801) as a white powdered solid (19.9 mg, yield=92%). ¹H-NMR (D2O, 600 MHz, T=5° C.) δ ppm: 1.68 (m, 2H, CONHCH₂CH₂CH₂NHCO), 2.09 (d, 1H, CHH), 2.21-2.34 (m, 5H, 2×CH, and CHH), 2.54-2.65 (m, 7H, 3×CH₂ and CHH), 2.97 (d, 1H, CH), 3.09 (d, 2H, CH₂), 3.17-3.42 (m, 6H, 3×CH₂), 3.44-3.58 (m, 4H, 2×CH₂), 3.63 (m, 2H, CH₂), 4.37 (d, 2H, —CH₂-guanidine), 7.44 (m, 2H, CH benzene ring), 7.61 (m, 2H, CH benzene ring). 13C-NMR (D2O, 150 MHz, T=5° C.) δ ppm: 180.25, 179.70, 175.82, 169.96, 156.56, 136.84, 133.21, 130.62, 129.15, 129.13, 126.17, 125.22, 65.45, 65.30, 65.21, 63.02, 55.63, 55.45, 55.43, 55.35, 54.74, 54.71, 54.57, 54.16, 43.75, 39.18, 38.39, 29.95.

HR-MS (ESI): calculated mass for [C₂₈H₄₂N₉O₈Y+H]⁺=722.2293 m/z; determined mass: 722.2284 m/z. Source parameters: capillary temperature 275° C.; spray voltage 3.70 kV; sheath gas 6; tube lens voltage 125 V. Resolution input: 100000. Samples dissolved in MeOH (10⁻⁵ M).

Chelate 10b′ (MC4803).

¹H-NMR (D₂O, 600 MHz, T=5° C.) δ ppm: 1.56 (m, 4H, CONHCH₂CH₂CH₂CH₂NHCO), 2.07 (d, 1H, CHH), 2.21-2.32 (m, 5H, 2×CH, and CHH), 2.54-2.65 (m, 7H, 3×CH, and CHH), 2.98 (d, 1H, CHH), 3.07 (d, 2H, CH₂), 3.16-3.42 (m, 6H, 3×CH₂), 3.44-3.59 (m, 4H, 2×CH₂), 3.65 (m, 2H, CH₂), 4.37 (d, 2H, —CH₂-guanidine), 7.44 (m, 2H, CH benzene ring), 7.62 (m, 2H, CH benzene ring). 13C-NMR (D₂O, 150 MHz, T=5° C.) δ ppm: 180.30, 179.70, 175.82, 169.97, 156.53, 136.85, 133.22, 130.65, 129.12, 129.10, 126.21, 125.20, 65.46, 65.31, 65.23, 63.05, 55.64, 55.44, 55.42, 55.36, 54.71, 54.70, 54.59, 54.19, 43.77, 39.21, 38.42, 27.17, 27.10.

HR-MS (ESI): calculated mass for [C₂₉H₄₄N₉O₈Y+H]⁺=736.2450 m/z; determined mass: 736.2442 m/z. Source parameters: capillary temperature 275° C.; spray voltage 3.70 kV; sheath gas 6; tube lens voltage 125 V. Resolution input: 100000. Samples dissolved in MeOH (10⁻⁵ M).

General procedure for the preparation of the chelates (radio-marking) of Formula III (10a, MC4801) and IV (10b, MC4803). Example: chelate 10a (MC4801). The chelate of Formula III (10a) has been prepared by adding 30 μl of a commercial solution of 90YCl_{b} in 0.05 M HCl (1.07 mCi, 22.1 μmol) a 132 μl of a 0.1 mM binder 13a solution (13.2 nmol) in 1M ammonium acetate buffer (pH=5.5-6) and bringing to the final volume of 500 μl by the addition of 338 μl of the same 1M ammonium acetate buffer (pH=5.5-6). The resulting solution has then been incubated in closed vial placed on a heating block under shielded hood at 90° C. for 30 minutes. At the end of the reaction, after cooling to room temperature, different aliquots of the solution (2-10 μl) have been collected without dilution to evaluate the radio-marking yield and to carry out the quality control by ITLC. The ITLC methods used ITLC-SG and ITLC-SA Agilent plates and different eluent systems such as 1M ammonium acetate buffer (pH=5.5-6):methanol (50:50 v/v), 1M ammonium acetate buffer (pH=5.5-6):methanol:ammonia 33% (50:50:5 v/v/v) and 50 mM EDTA in 0.1 M ammonium acetate buffer (pH=6). The results of different chromatographic runs showed in agreement >99% radio-marking yield and radiochemical purity.

Chemical Stability of the Chelates Containing ⁸⁹Y³⁺ [Compounds of Formula III (10a′, MC4801) and IV (10b′, MC4803)] as Non-Radioactive Analogues of the Chelates of Formula III and IV (10a,b or MC4801 and MC4803) in Physiological Conditions.

The chemical stability of the non-radioactive analogues of the chelates of Formula III and IV, (10a′,b′ or MC4801 and MC4803) in physiological conditions has been evaluated by analysis with analytical HPLC (Shimadzu Nexera chromatograph equipped with a SPD-M20A PDA detector; Hypersil ODS GOLD 250×4.6 mm column; eluent: MeOH/H₂O 5:95 v/v+0.02% TFA, flow: 1.0 ml/min; UV detection 214 nm) repeated at regular time intervals after having solubilized it in PBS buffer (c=0.9 mg/ml) and kept at T=37° C. After 5 days, both chelates were found to be still perfectly intact, with a chemical purity constantly higher than 99.5%.

Biological Validation of the Chelates Containing ⁸⁹Y³⁺ [Compounds of Formula III (10a′, MC4801) and IV (10b′, MC4803)] as Non-Radioactive Analogues of the Chelates of Formula III (10a, MC4801) and IV (10b, MC4803) as Substrates of NET in the Human Neuroblastoma Line SK-N-SH.

The ability of the “cold” chelates of Formula III (10a′, MC4801) and IV (10b′, MC4803) to act as substrates of NET has been evaluated in competition experiments with the tritiated endogenous substrate ³H-NE for the uptake by the human neuroblastoma cells SK-N-SH, which are known to express the transporter NET in large quantity. In this cell line, both chelates 10a′,b′ (MC4801 and MC4803) showed dose-dependent inhibition of the uptake/internalization of ³H-NE, showing to be a substrate of NET analogously to MIBG, even though with lower power (FIG. 4) and without dose-dependent cytotoxic effects which are evident up to the maximum concentration tested (100 μM). It is interesting to note how, also in this case, analogously to what already observed with the free binder 9 (MC4325) of the compound of Formula II (MC4324), the free binders 13a,b (denoted as MC4802 and MC4804, respectively) of the compounds of general formula III (MC4801) and IV (MC4803) were found to be completely unable to compete with ³H-NE for the internalization up to the higher concentration tested (FIG. 4). Therefore also in this case it is possible to prepare radio-marked versions of the chelates of Formula III (10a, MC4801) and IV (10b, MC4803) with low specific activity, as the absence of competition for NET between the chelates 10a,b (MC4801 and MC4803) and the respective free binders 13a,b (MC4802 and MC4804) doesn't compromise the bonding ability to NET of the chelates [compounds of Formula III (10a, MC4801) and IV (10b, MC4803)] also in the presence of an excess of free binders 13a,b (MC4802 and MC4804).

FIG. 4 depicts the competition of the “cold” chelates of Formula III (10a′, MC4801) and IV (10b′, MC4803) and respective free binders 13a,b (MC4802 and MC4804) with ³H-NE (50 nM, 1 hour of incubation, T=37° C.) for the uptake by the SK-N-SH cells. ¹H-NE at 20 μM and MIBG at 2 μM have been used in analogous competition experiments as positive controls.

Materials and Methods Relative to the Cell Tests

Cell Lines and Culture Conditions

The human neuroblastoma cell line SK-N-SH has been purchased from ATCC. The cells have been kept in E-MEM medium containing 10% fetal bovine serum (FBS), 2 mM L-glutamine and antibiotics in humidified atmosphere with 5% CO₂ at 37° C.

Cell Uptake of ³H-NE

³H-NE has been purchased from Perkin Elmer. To measure the initial cell uptake speeds of ³H-NE, the cells have been grown for 16 hours in the presence of serum only, then incubated with binder medium (EMEM containing 0.2% BSA and 20 mM Hepes, pH 7.5) for 10 minutes by heating in a water bath at 37° C. The bond has been initiated by adding 0.5 ml per well of 50 nM ³H-NE to the binder medium for a long time, the plates have then been placed on ice and washed three times with frozen PBS. Thus, the cell monolayers have been dried and lysed with 2% NaOH 1N SDS. The internal cpm (“counts” per minute) have been determined separately in the different wells in triple for each time. The background bond has been determined in parallel in a fourth well containing a 400 times molar excess of non-marked ¹H-NE (“cold”). For the competition study, the cells have been plated as above, and thus incubated for 1 hour with 50 nM ³H-NE in binder medium in the absence and presence of the “cold” chelates of Formula III (10a′, MC4801) and IV (10b′, MC4803) and respective free binders 13a,b (MC4802 and MC4804, respectively) at different concentrations. The radioactivity has been determined separately in the wells in triple for each concentration value. The uptake of ³H-NE has been expressed as total radioactivity percentage normalized for mg of protein.

Stability in Serum of the Chelates of Formula III (10a, MC4801) and IV (10b, MC4803).

The stability in serum of the chelates of Formula III (10a, MC4801) and IV (10b, MC4803) has been evaluated by measuring the release of the metal cation ⁹⁰Y³⁺ from the chelates to the serum proteins during 14 days in which they have been kept in physiological conditions. Briefly, the chelates of Formula III (10a, MC4801) and IV (10b, MC4803) have been incubated with human serum (32 MBq per 16 ml of serum) at T=37° C. At regular time intervals serum aliquots have been collected and, by using centrifuge filter tubes (Amicon® Ultra-4 3K, Merck Millipore) and by centrifuging at 5500 g, the serum proteins have been separated from the non-protein fraction of serum and the radioactivity of both fractions has been measured with a liquid scintillation β counter (scintillation liquid used is Perkin Elmer ULTIMA GOLD). During the 14 days of observation no measurable loss of radioactivity (⁹⁰Y³⁺) in favor of the protein fraction of serum has been detected.

### BRIEF DESCRIPTION OF THE FIGURES

FIG. 1 depicts the ITLC-SG runs relative to the radio-marking of the binder 9 with ⁹⁰YCl₃.

FIG. 2 depicts the HPLC plots relative to the evaluation of the chemical stability over time of the compound ⁸⁹Y-DOTA-BG (1a).

FIG. 3 depicts the competition of the chelate of Formula II (1a, MC4324) and the respective free binder 9 (MC4325) with ³H-NE for the uptake by the neuroblastoma cells SK-N-SH

FIG. 4 depicts the competition of the chelates of Formula III (10a′, MC4801) and IV (10b′, MC4803) and respective free binders 13a (MC4802) and 13b (MC4804) with ³H-NE for the uptake by the neuroblastoma cells SK-N-SH

### DETAILED DESCRIPTION OF THE FIGURES

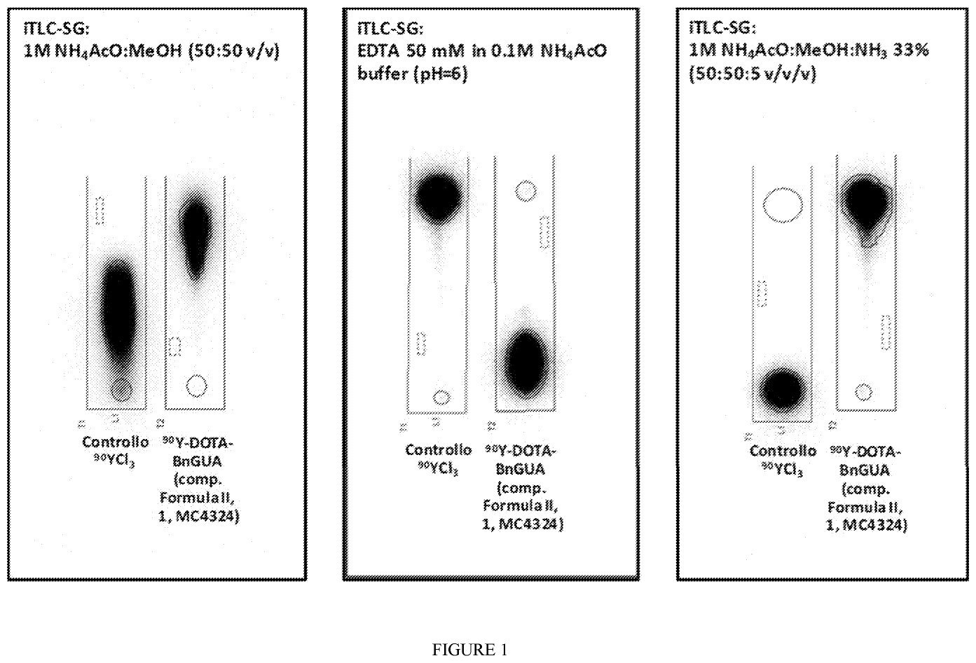

FIG. 1 depicts the ITLC-SG runs relative to the radio-marking of the binder 9 with ⁹⁰YCl₃ for the preparation of the chelate ⁹⁰Y-DOTA-BG of Formula II (1, or MC4324).

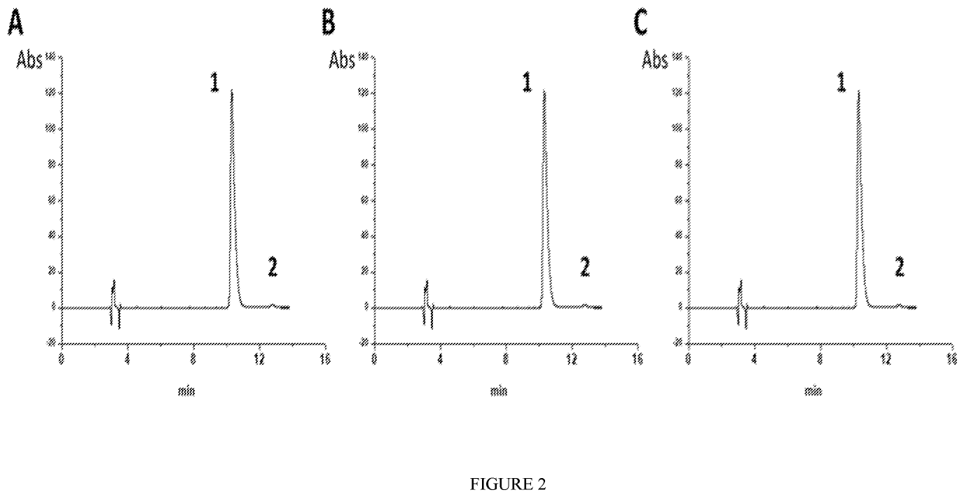

FIG. 2 depicts the HPLC plots relative to the chemical stability over time of the ⁸⁹Y-DOTA-BG chelate (1a) solubilized in PBS buffer (c=0.9 mg/ml). FIG. 2A depicts the plot of ⁸⁹Y-DOTA-BG (1a) solubilized in PBS buffer at T=37° C. and time t=0 min. FIG. 2B depicts the plot of ¹¹¹Y-DOTA-BG (1a) solubilized in PBS buffer at T=37° C. and time t=21 hours. FIG. 2C depicts the plot of ⁸⁹Y-DOTA-BG (1a) solubilized in PBS buffer at T=37° C. and time t=5 days.

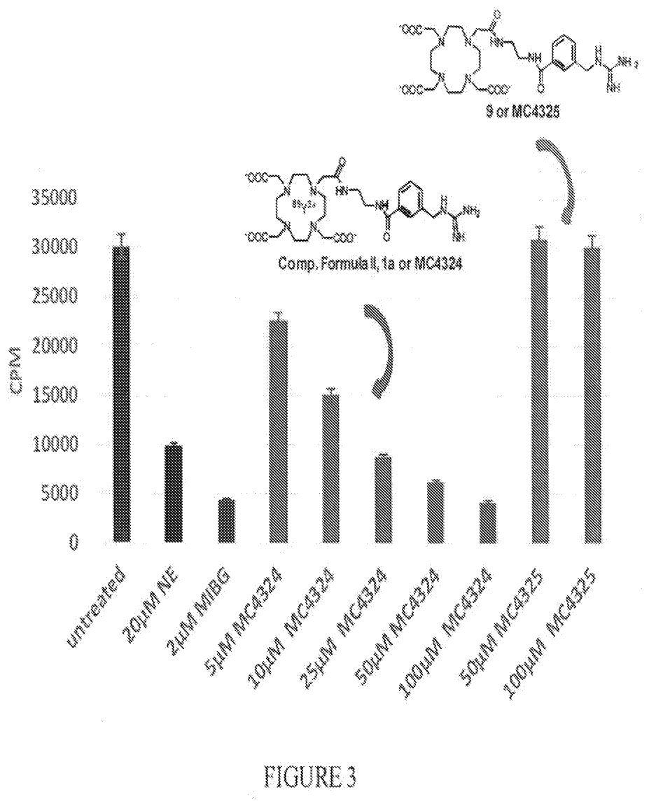

FIG. 3 depicts the results of the competition assay of the chelate of Formula II (1a, MC4324) and respective free binder 9 (MC4325) with ³H-NE for the uptake/internalization through NET by the human neuroblastoma cells SK-N-SH. The first column of FIG. 3 starting from the left is relative to the non-treated control (lack of competition). The following two columns are relative to two positive controls, the ¹H-NE itself (20 μM) and the MIBG (2 μM) both non-marked which compete with ³H-NE for the internalization at the depicted doses. From the fourth to the eighth column in FIG. 3 the results of the competition experiments with increasing doses (5 to 100 μM) between the chelate of Formula II (1a, MC4324) and ³H-NE are depicted. In the ninth and tenth column in FIG. 3 the results of the competition experiments at two different concentrations (50 and 100 μM) between the free binder of the compound of Formula II (9 or MC4325) and ³H-NE are reported.

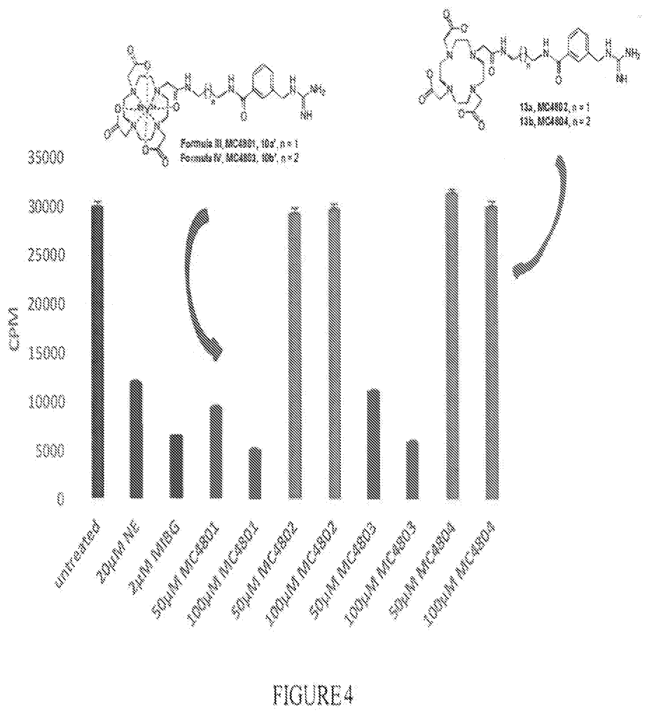

FIG. 4 depicts the results of the competition assay of the chelates of Formula III (10a′, MC4801) and IV (10b′, MC4803) and the respective free binders 13a (MC4802) and 13b (MC4804) with ³H-NE for the uptake/internalization through NET by the human neuroblastoma cells SK-N-SH. The first column of FIG. 4 starting from the left is relative to the non-treated control (absence of competition). The following two columns are relative to two positive controls, the ¹H-NE itself (20 μM) and the MIBG (2 μM) both non-marked which compete with ³H-NE for the internalization at the depicted doses. The fourth and fifth column in FIG. 4 depict the results of the competition experiments between the chelate of Formula III (10a′, MC4801) at two different concentrations (50 and 100 μM) and ³H-NE. The sixth and seventh column in FIG. 4 depict the results of the competition experiments between the free binder of the chelate of Formula III (13a, MC4802) at two different concentrations (50 and 100 μM) and ³H-NE. The eighth and ninth column in FIG. 4 depict the results of the competition experiments between the chelate of Formula IV (10b′, MC4803) at two different concentrations (50 and 100 μM) and ³H-NE. The tenth and eleventh column in FIG. 4 depict the results of the competition experiments between the free binder of the chelate of Formula IV (13b, MC4804) at two different concentrations (50 and 100 μM) and ³H-NE.

1. A compound of Formula I 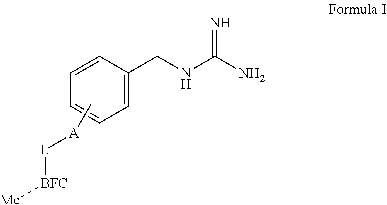
wherein
A is an anchoring portion chosen from the group consisting of amide, anilide, ether, amine and sulfonamide, linked to the meta or para position of benzylguanidine (BG);
L is a linker portion chosen from the group consisting of diaminoalkyl chains with lengths ranging from 2 to 6 methylene units and diaminopolyethylene glycol chains with lengths ranging from 2 to 6 ethylene glycol units;
BFC is a bifunctional chelator chosen from the group consisting of DOTA, NOTA, TETA and DTPA; and
Me is a radiation-emitting or non-radiation-emitting metal cation chosen from the group consisting of ⁹⁰Y^{3+ 177}Lu³⁺, ⁸⁶Y³⁺, ⁸⁹Y³⁺, ⁶⁸Ga³⁺ and ¹¹¹In³⁺.

2. The compound according to claim 1, wherein said metal cation is a pure β⁻ radiation-emitting metal cation.

3. The compound according to claim 1, wherein said bifunctional chelator BFC is DOTA.

4. The compound according to claim 1, wherein that that said linker portion L is an ethylenediamine group.

5. The compound according to claim 1, wherein said anchoring portion A is an amide group.

6. The compound according to claim 1 of Formula II: 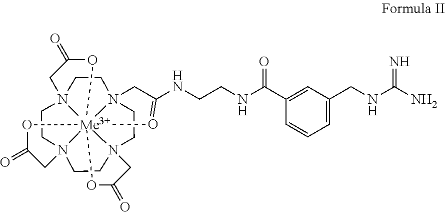
wherein Me is a radiation-emitting or non-radiation-emitting metal cation chosen from the group consisting of ⁸⁶Y³⁺, ⁸⁹Y³⁺, ⁶⁸Ga³⁺, ¹¹¹In³⁺, ¹⁷⁷Lu³⁺ and ⁹⁰Y³⁺, preferably it is ⁹⁰Y³⁺ or ⁸⁹Y³⁺.

7. The compound according to claim 1, having the following Formula III: 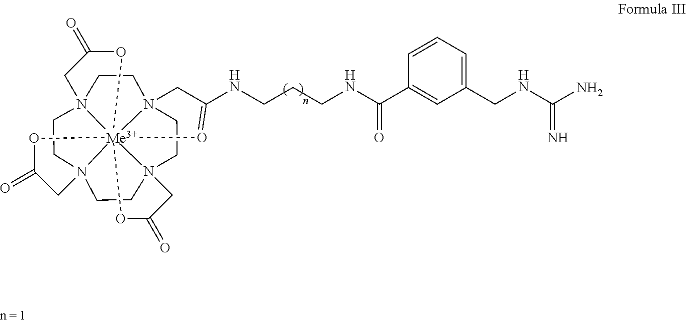
wherein n is equal to 1 and wherein Me is a radiation-emitting or non-radiation-emitting metal cation selected from the group consisting of ⁸⁶Y³⁺, ⁸⁹Y³⁺, ⁶⁸Ga³⁺, ¹¹¹In³⁺, ¹⁷⁷Lu³⁺ and ⁹⁰Y³⁺.

8. The compound according to claim 1 having the following formula IV: 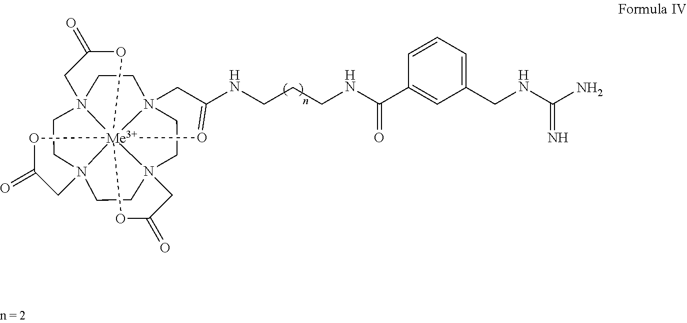
wherein n is equal to 2 and wherein Me is a radiation-emitting or non-radiation-emitting metal cation selected from the group consisting of ⁸⁶Y³⁺, ⁸⁹Y³⁺, ⁶⁸Ga³⁺, ¹¹¹In³⁺, ¹⁷⁷Lu³⁺ and ⁹⁰Y³⁺.

9. A medicament comprising the compound according to claim 1.

10. Method of β radio-tracing with the compound according to claim 1 in a subject having a tumor, said method comprising
administering to said subject an effective amount of said compound.

11. A method of treating or diagnosing a subject having a tumor with the medicament according to claim 9, wherein said tumors are selected from the group consisting of neuroendocrine tumors which overexpress the norepinephrine transporter (NET), pheochromocytoma, paraganglioma, carcinoid tumor and neuroblastoma.

12. Method of radio-guided surgery (RGS) with the medicament according to claim 9.

13. The method according to claim 12, wherein the tumor surgery is radio-guided by the β⁻ particle detection (β⁻-RGS).

14. A pharmaceutical composition comprising the compound according to claim 1 and pharmaceutically suitable excipients.

15. The compound according to claim 7, wherein Me is ⁹⁰Y³⁺ or ⁸⁹Y³⁺.

16. The compound according to claim 8, wherein Me is ⁹⁰Y³⁺ or ⁸⁹Y³⁺.
